(19) United States
(12) Patent Application Publication
(10) Pub. No.: US 2008/0194970 A1
(43) Pub. Date: Aug. 14, 2008

(54) Methods for Intraoperative Organotypic Nerve Mapping

(76) Inventors: William D. Steers, Charlottesville, VA (US); Lisa Blackburn Boyette, Arlington, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/997,978

(22) PCT Filed: Aug. 16, 2006
(86) PCT No.: PCT/US06/32028

§ 371 Date: Feb. 5, 2008

Related U.S. Application Data
(60) Provisional application No. 60/709,872, filed on Aug. 19, 2005.

(51) Int. Cl. A61B 5/04

(52) U.S. Cl. 600/476

(57) ABSTRACT

The present invention relates to compositions, methods and apparatuses for locating a nerve during a surgical procedure.

### CROSS REFERENCE TO RELATED APPLICATIONS

This application is entitled to priority pursuant to 35 U.S.C. §119(e) to U.S. provisional patent application No. 60/709,872, filed on Aug. 19, 2005, the entirety of which is incorporated by reference herein.

### FIELD OF THE INVENTION

The present invention relates to compositions, methods, and apparatuses for locating a nerve during a surgical procedure. The invention also relates to preserving a nerve from injury during a surgical procedure.

### BACKGROUND

Studies have shown that as many as two thirds of men develop erectile dysfunction (ED) following radical prostatectomy for prostate cancer or radical cystectomy for bladder cancer as a result cavernous nerve injury during surgery. The cavernous nerves are small autonomic nerve fibers that course along the capsule of the prostate to the corpus cavernosum of the penis. These nerves are responsible for penile erection. Potency rates (i.e., cavernous nerve preservation) vary dramatically among surgeons based on experience, patient factors (age, difficult anatomy, and comorbidities such as diabetes mellitus), tumor biology, and whether cavernous nerves have been intentionally preserved. Experienced surgeons rely on gentle tissue handling and estimating the location of these microscopic nerves. However, individual variability in the course of nerves as well as possibility of cancer extension into the neurovascular bundle or surrounding tissues exists. Thus, the need for a method to reliably identify these nerves and extraprostatic extension of cancer is dire, given that 37,000 radical prostatectonies are performed annually in increasing numbers of younger men (see generally, Anastasiadis et al., Urology, 62:2:292-7, 2003; Potosky et al., J. Natl. Cancer Institute, 92:19:1582-92, 2000; Penson et al., J. Urol. 173:5:1701-5, 2005; Ward et al., J. Urol. 172:1328-32, 2004).

Among patients undergoing robotic assisted laparoscopic radical prostatectomy in whom visualization is improved and blood loss less, ED has been reported in anywhere from 35-75% of men. Identifying methods for preserving nerve function are imperative for improving patient outcomes from these surgeries (for example, see U.S. Pat. No. 5,284,153). Current use of imaging technology during surgery is generally limited to ultrasound, PET, open MRI or other expensive, cumbersome devices that lack adequate cellular resolution for fine tumor border localization and organ specific nerve identification. Often images are registered with pre-computed or sensed data to provide a map during in vivo procedures rather than data acquired in real time. Optical imaging on the cellular level has been restricted to in vitro applications due to problems with optical access or labeling. New technologies such as optical coherence tomography rely on infrared reflections from interfaces several millimeters deep in tissues with a resolution of tens of micrometers to provide a gray scale histologic image. Some investigators have tried staining for NADPH found in nerves to the penis although this methodology fails to distinguish penile, urethral, or prostatic nerves.

There is a long felt need in the art for methods and apparatuses useful for identifying and sparing damage to nerves during surgical procedures. The present invention satisfies this need.

### BRIEF SUMMARY OF INVENTION

The present invention provides a real time method for visualizing nerves in vivo. In one aspect, the invention provides for visualizing cavernous nerves in vivo. The present invention further provides a method to aid surgeons in nerve preservation and tumor border identification during robotic radical prostatectomy and during other surgical procedures where it is important to identify and preserve nerves.

Because of the recent commercial availability of portable, fluorescent, fiber-optic laser confocal microscopes, it is now practical to employ the methods of the present invention for acquiring repeated microscopic images of living tissues of interest with minimal damage to these tissues. The unique high-sensitivity design of the present invention allows imaging of structures as small and as difficult to locate as the fine autonomic nerves fibers to internal organs. In one aspect, when used intra-operatively with the da Vinci robotic platform to simultaneously identify cavernous nerves (to avoid injury resulting in erectile dysfunction (ED)) and prostate cancer (to prevent the occurrence of positive tumor margins); it is envisioned that this technique will revolutionize surgery, allowing the possibility of molecular and cellular surgery rather than gross anatomical surgery, thereby reducing cancer recurrences and reducing morbidity associated with surgery.

The present invention provides compositions, methods, and apparatuses for labeling and then visualizing cavernous nerves in real time during radical prostatectomy.

Similar methods for identifying prostate cancer and other cancers are encompassed within the present invention. Using fluorescent retrograde nerve tracers, cavernous nerves have been labeled herein and subsequently imaged intra-operatively using a fiber optic laser confocal fluorescence microscope. Other visualization methods can also be practiced with the present invention.

In one embodiment of the invention, fluorescent retrograde tracer labels are injected into axons into the corpus cavernosum of an awake, unanesthetized subject. In one aspect, up to about a 24 hour period is allowed for transport of the tracer to the sites of interest near the prostate, and then a small fiber-optic microscope probe of high sensitivity is used to measure fluorescence while dissection of the area surrounding the prostate is performed, either in an open or closed procedure. In another aspect, up to about several days is allowed for transport of the tracer.

One of ordinary skill in the art will appreciate that other tracers can be used, depending on the particular surgical procedure being performed, the instrumentation being used, and on variables such as age, health, and weight of the subject. One of ordinary skill in the art will also appreciate that depending on the particular tracer being used, the time needed for visualizing the tracer may vary. In one aspect, the time in which the tracer is available for visualization may vary after its introduction to the subject. In one aspect, more than one tracer may be used. Most retrograde neuronal tracers label neuronal cell bodies, this invention employs tracers optimized to identify axons and nerve bundles, which are more relevant to surgical identification in humans.

Clinically, in one aspect, the present invention encompasses injection of tracer into the penis at about 24 hours prior to surgery. In one aspect, one or both corpora cavernosa are injected. In another aspect, one or both crura of the penis are injected. In yet another aspect, the corpus spongiosum is injected. In a further aspect, other tissues in the area are injected. It will be appreciated by one of ordinary skill in the art that combinations of these sites could also be injected. The invention further encompasses injecting various amounts of tracer, as well as injecting at different amounts of time prior to surgery. Then, during robotic-assisted laparoscopic prostatectomy or cystectomy the Cell Vizio device would be mounted on a robotic stabilizer (arm) and brushed along side the neurovascular bundle during dissection to provide a road map to safely avoid cauterizing or applying traction on these fine nerve fibers. The novelty of the technique is derived from: 1) the use of retrograde tracers to positively identify exactly those nerves (axons as opposed to cell bodies) supplying the penis that control penile erection that course beside the prostate; and 2) employing fiber optic laser confocal fluorescence microscopy to non-invasively, reversibly image these nerves intra-operatively. This process allows viewing labeled axons and not connective or vascular tissues that obscure the nerves without altering the function of the cavernous nerves. This unique method of labeling and imaging during surgery makes it possible for us to detect the nerves at various levels during the surgery, thus avoid damaging them.

One of ordinary skill in the art would appreciate that markers and tracers exist other than the ones described herein and would be useful in the practice of the invention.

In one embodiment, the present invention is useful for toxicity studies of AlexaFluor-488 coupled to Cholera Toxin Subunit B.

In one embodiment, the present invention is useful for studying erectile function and apoptosis studies following AlexaFluor-488-conjugated Cholera Toxin Subunit B injections and fluorescent imaging using cavernous nerve stimulation and intracavernous pressure measurements.

In another embodiment, the invention is useful for feasibility studies of voltage and calcium (Ca⁺²) sensitive dyes and in vivo markers for reactive oxygen species (ROS) (see FIG. 5) during electrocautery and traction to detect transients indicating nerve activation or possible injury during surgery.

In yet another embodiment, the invention is useful for fibered confocal fluorescent microscopy studies of prostate after systemic injection of fluorescent yellow protein tagged to a PSMA antibody or an antagonist to detect the prostate border.

In a further embodiment, the invention is useful for fibered confocal fluorescent microscopy studies of prostate cancer in transgenic mouse models of prostate cancer.

In another embodiment, the invention is useful for fibered confocal fluorescent microscopy studies of ex vivo human prostate injected with fluorescent yellow protein-labeled PSMA antagonist.

In another embodiment, the invention is useful for following toxicology and safety studies and for use in clinical studies with this nerve mapping technique during robotic surgery for prostate and bladder cancer. Feasibility of labeling will be combined with outcome studies for preservation of erectile function using pre- and post-operative International Index of Erectile Function (IIEF) instrument.

In yet another embodiment, the invention is useful for clinical feasibility studies of fluorescently-tagged PSMA antagonist or PSMA antibody administered systemically with fibered confocal fluorescent microscopic imaging of prostates during robotic surgery.

### BRIEF SUMMARY OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 represents an image of a gross light photograph of rat corpus cavernosum; the area injected with retrograde tracers has been stained with FastBlue (appears black in photo).

FIG. 2 represents a photographic image of cavernous nerve labeled with AlexaFluor-488-conjugated Cholera Toxin Subunit B. This in vivo image was obtained from a live rat during a surgical procedure using fibered confocal fluorescence microscopy. The marker bar indicates 30 microns.

FIG. 3 represents a photographic image of normal prostate cells labeled with topical acriflavine in a rat during a surgical procedure and was obtained using fibered confocal fluorescence microscopy. The marker bar indicates 30 microns.

FIG. 4 is a schematic representation of a flow diagram summarizing nine processes and methods for tracing the path of a neurovascular bundle during surgery.

FIG. 5, comprising eight panels of images, shows peri-prostatic tissue after as long as 5 minutes of continuous laser imaging demonstrating no increase in reactive oxygen species, implying the technique is safe and does not damage tissues. Control (top panels) panel is no laser imaging, then 2 (second from top) and 5 (third panels) minutes of imaging. Lastly compound given to cause ROS as positive control (lower panels).

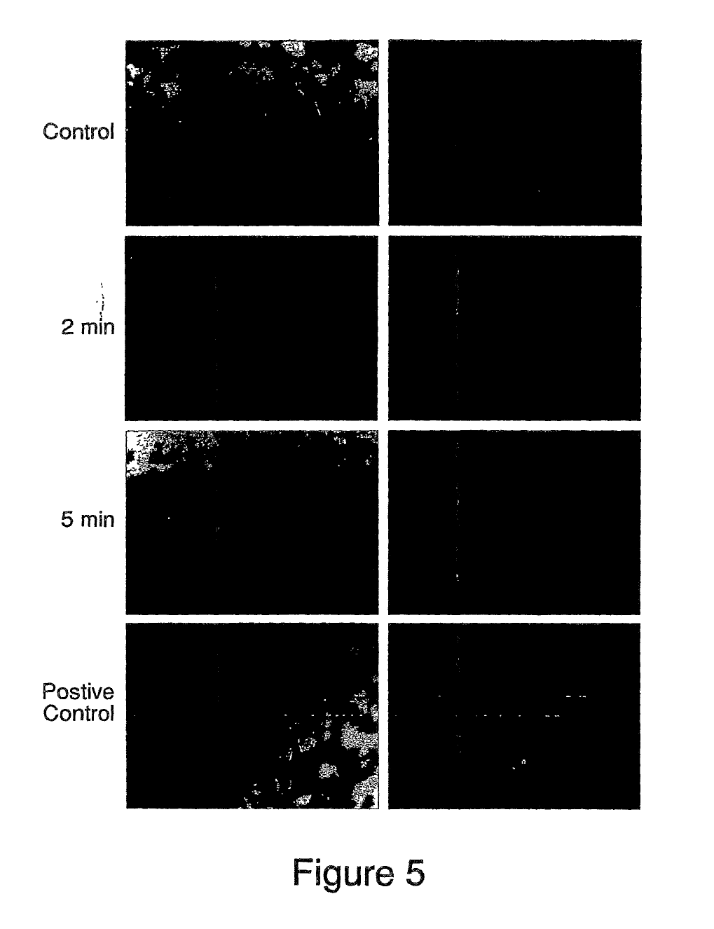

### DETAILED DESCRIPTION OF THE INVENTION

### Abbreviations and Acronyms

ED—erectile dysfunction
IIEF—International Index of Erectile Function
PSMA—prostate specific membrane antigen

### Definitions

In describing and claiming the invention, the following terminology will be used in accordance with the definitions set forth below.

As used herein, the articles “a” and “an” refer to one or to more than one, i.e., to at least one, of the grammatical object of the article. By way of example, “an element” means one element or more than one element.

As used herein, the term “affected cell” refers to a cell of a subject afflicted with a disease, injury, or disorder, which affected cell has an altered phenotype relative to a subject not afflicted with a disease or disorder.

Cells or tissue are “affected” by a disease, injury, or disorder if the cells or tissue have an altered phenotype relative to the same cells or tissue in a subject not afflicted with a disease or disorder.

A disease or disorder is “alleviated” if the severity of a symptom of the disease, condition, or disorder, or the frequency with which such a symptom is experienced by a subject, or both, are reduced.

“Analgesia” is defined as a condition in which nociceptive stimuli are sensed but are not interpreted as pain. “Anesthesia” is a state characterized by total loss of sensation, the result of pharmacologic depression of nerve function. Thus, analgesia does not produce anesthesia whereas anesthesia produces analgesia.

As used herein, an “analog” of a chemical compound is a compound that, by way of example, resembles another in structure but is not necessarily an isomer (e.g., 5-fluorouracil is an analog of thymine).

The term “cancer,” as used herein, is defined as proliferation of cells whose unique trait—loss of normal controls—results new characteristics such as unregulated growth, lack of differentiation, local tissue invasion, and metastasis. Examples include, but are not limited to, breast cancer, prostate cancer, ovarian cancer, cervical cancer, skin cancer, melanoma, pancreatic cancer, colorectal cancer, renal cancer, leukemia, non small cell carcinoma, and lung cancer.

A “compound,” as used herein, refers to any type of substance or agent that is commonly considered a drug, or a candidate for use as a drug, as well as combinations and mixtures of the above.

As used herein, a “derivative” of a compound refers to a chemical compound that may be produced from another compound of similar structure in one or more steps, as in replacement of H by an alkyl, acyl, or amino group.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, an “effective amount” means an amount sufficient to produce a selected effect.

The terms “formula” and “structure” are used interchangeably herein.

As used herein, “homology” is used synonymously with “identity.”

The phrase “identifying a nerve”, as used herein refers to identifying small branches of nerves or nerve plexuses. The term “identifying” includes, but is not limited to, various methods of visualizing or imaging small nerve fibers and plexuses, or fibers within a plexus.

The term “identifying a nerve at a site of interest” refers to visualizing a nerve in a particular location, particularly small nerve fibers, in an area such as the nerve plexus around the prostate gland where the cavernous nerves traverse.

The term “inhibit,” as used herein, refers to the ability of a compound of the invention to reduce or impede a described function, such as having inhibitory sodium channel activity. Preferably, inhibition is by at least 10%, more preferably by at least 25%, even more preferably by at least 50%, and most preferably, the function is inhibited by at least 75%. The terms “inhibit” and “block” are used interchangeably herein.

As used herein, the term “imaging agent” means a composition of matter which, when delivered to a cell or tissue, facilitates detection of the cell or tissue. Numerous imaging agents are known and described in the literature. By way of example, enzymes, such as β-galactosidase, which are capable of catalyzing a reaction involving a chromogenic substrate may be used. Further by way of example, compounds, the presence of which may be directly detected may be used, such as compounds which emit gamma radiation or which fluoresce, which may be detected using an appropriate detection apparatus.

A “reversibly implantable” device is one which may be inserted (e.g. surgically or by insertion into a natural orifice of the animal) into the body of a subject and thereafter removed without great harm to the health of the subject.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal, including the surgical process, as well as administering the labeled markers. The instructional material of the kit of the invention may, for example, be affixed to a container which contains one or more compounds useful in the invention or be shipped together with a container which contains the identified compound. Alternatively, the instructional material may be shipped separately from the container with the intention that the instructional material and the compound be used cooperatively by the recipient.

A “natural” orifice of an animal is an orifice (e.g. a mouth, nostril, epidermal pore, anus, etc.) which is normally present in an animal which is not afflicted with a disease or disorder.

A “non-naturally-occurring” orifice of an animal is an orifice (e.g. an incision, puncture, wound, etc.) which is not normally present in an animal which is not afflicted with a disease or disorder.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “photoactivation” and grammatical forms thereof refer to a process by which, upon absorption of a quantum of energy corresponding to a photon of light having a given wavelength, a chemical compound is enabled to participate in or undergo a chemical reaction at a reaction rate which is greater than the corresponding reaction rate in the absence of photoactivation.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “revealed by a surgical procedure” means that site of interest for localization or visualization of a nerve must be approached and revealed with some sort of surgical procedure, such as laparoscopy.

The term “standard,” as used herein, refers to something used for comparison. For example, it can be a known standard agent or compound which is administered or added and used for comparing results when adding a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of diagnosis or treatment is a mammal, including a human.

The term “surgical procedure” as used herein, refers to use of the invention before a surgical procedure as well as during a surgical procedure.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

A tissue “normally comprises” a cell if one or more of the cells are present in the tissue in an animal not afflicted with a disease or disorder.

An “internal” tissue of an animal is a tissue which is normally located beneath the epidermis of the animal, within the animal's body.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “tracer”, as used herein, refers to a molecule that is conveyed along or within a nerve and is labeled with a substance which can be visualized or imaged. The tracer may or may not need to be stimulated (i.e., by light) to be visualized or localized.

As used herein, the term “treating” includes prophylaxis of the specific disease, disorder, or condition, or alleviation of the symptoms associated with a specific disease, disorder, or condition and/or preventing or eliminating said symptoms.

In one embodiment, the present invention provides for intraoperative identification of functional nerves. In one aspect, the invention provides for identification of nerves in virtually any anatomical location. In one aspect, the technique is useful during intraoperative procedures performed on humans. In another aspect, the procedure is useful for nerve identification and tracing in experimental animal work.

In one embodiment, the present invention provides for identification of functional nerves during surgical procedures, as well as concomitant treatment with drugs or other procedures.

In conjunction with the use of the fluorescent labeling of the nerves as disclosed herein, the present invention can be used with other techniques as well. For example, the labeled nerves could also be stimulated, such as along the prostate, to determine whether there is a rise in penile pressure. Additionally, non-specific visualization can be used employing a stain that labels all nerves.

Techniques useful for carrying out the present invention are known to those of ordinary skill in the art (see for example U.S. Pat. Nos. 5,284,153 and 5,284,154; U.S. Pat. Pub. No. 20030228260; Anastasiadis et al., Urology, 62:2:292-7, 2003; Potosky et al., J. Natl. Cancer Institute, 92:19:1582-92, 2000; Penson et al., J. Urol. 173:5:1701-5, 2005; Ward et al., J. Urol. 172:1328-32, 2004). The anatomy of the penis, prostate, and bladder regions is known in the art (see, e.g., Williams et al., eds., 1980, Gray's Anatomy, 36th ed., W. B. Saunders Co., Philadelphia, Pa., USA).

To detect a molecule of interest, a label can be used. The label can be coupled to a binding antibody or other interacting polypeptide, or to one or more particles, such as a nanoparticle. Suitable labels include, but are not limited to, fluorescent moieties, such as fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3 and 5; phycoerythrin; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives; pyrenes; pyridyloxazole derivatives; dapoxyl dyes; Cascade Blue and Yellow dyes; benzofuran isothiocyanates; sodium tetrafluorophenols; 4,4-difluoro-4-bora-3a,4a-diaza-s-indacene. In some cases enzymatic moieties can be appropriate, such as alkaline phosphatase or horseradish peroxidase; and radioactive moieties, including ³⁵[S] and ¹³⁵[I] labels. The choice of the label depends on the application, the desired resolution and the desired observation methods. For fluorescent labels, the fluorophore is excited with the appropriate wavelength, and the sample observed using a suitable microscope, such as a confocal microscope.

In addition to introducing stains and dyes into living cells, the methods of the invention also allow the introduction of nanoparticles that can be used as detectable entities in and of themselves (e.g., quantum dots) or to amplify a signal, whether innate to a target molecule or introduced. For example, metallic nanoparticles create surface-enhanced resonances, amplifying the natural fluorescence, auto-fluorescence, or fluorescently stained molecules by orders of magnitude. Using metallic nanoparticles therefore act as molecular mirrors, deflecting and augmenting available light signals to which they are in close proximity. The nanoparticles prevent energy loss of the stimulating radiation to other modes, like phonons, and ensure that the energy is channeled into emitted light. Because the natural fluorescence intensity of some target molecules, such as DNA, is normally very low, amplification the available signal reduces reliance on dyes or stains which can interfere with normal functioning of the target molecule. For example, many DNA-specific dyes intercalate between the bases; this intercalation can, in mitotically or meiotically active cells, introduce mutations into the genetic code.

Thus, one aspect of the present invention is a method of imaging a molecular event in a sample, the method steps comprising administering to the sample a molecule comprising a label/probe having the ability to be imaged in a nerve. In one aspect, the labeled molecule can be administered at one location, and its retrograde movement in the nerve can be monitored. The probe has at least one of a ligand/signaling agent combination, or conjugable form of a ligand/signaling agent combination. After the probe is administered, a signal from the probe may be detected. In embodiments of the present invention, the sample can be at least one of any tissue through which nerves, pass, particularly small nerve fibers.

Another aspect of the present invention is a method of quantifying the progression of a disease state progression.

The in vivo administration step may further comprises at least one time course imaging determination, and in other embodiments, the in vivo administration step further comprises at least one bio distribution determination, particularly where the administration is being performed in conjunction with a procedure to determine tumor boundaries.

For the purposes of the present invention, the term analog encompasses isomers, homologs, or other compounds sufficiently resembling the base compound in terms of structure and do not destroy activity. “Conjugable forms,” “conjugable compounds,” and similar terms describe a form of the compound that can readily form a covalent form a covalent bond with a signaling agent such as an IR or fluorescent dye.

Alexa Fluor 488 is used herein, although one of ordinary skill in the art will appreciate that other markers can be used as well. Alexa Fluor 488 dye is the best fluorescein (FITC or FAM) substitute available for most applications, particularly for single-molecule detection of bioconjugates, for fluorescence correlation spectroscopy (Technical Focus: Fluorescence Correlation Spectroscopy (FCS)) and for fluorescence polarization measurements (Technical Focus: Fluorescence Polarization (FP)). This green-fluorescent dye exhibits several unique features:

fluorescence spectra almost identical to those of fluorescein, with excitation/emission maxima of 495/519 nm and a fluorescence lifetime of ˜4.1 nanoseconds;

strong absorption, with an extinction coefficient greater than 65,000 cm−1M−1;

much greater photostability than fluorescein, allowing more time for observation and image capture; and

pH-insensitive fluorescence between pH 4 and 10;

water solubility, with no organic co-solvents required in labeling reactions;

superior fluorescence output per protein conjugate, surpassing that of any other spectrally similar fluorophore-labeled protein, including fluorescein conjugates; and

utility as a fluorescence anisotropy probe for measuring protein-protein interactions.

Alexa Fluor dyes exhibit higher fluorescence intensity than fluorescein and rhodamine, providing greater detection sensitivity. Alexa Fluor dyes are more photostable than fluorescein and rhodamine, so photobleaching is no longer a problem. Alexa Fluor® dyes are less sensitive to pH than are fluorescein and rhodamine, making the dyes useful over a broader pH range In one aspect, the invention provides a method of assisting diagnostic investigation or surgical treatment, said method comprising administering into a vascularized peripherally innervated tissue site, or into other tissue sites, a pharmaceutical composition comprising a labeled molecule which can be taken up in to a nerve cell capable of axonal-transport from said tissue site, and, where said method is to assist diagnostic investigation or surgical treatment, detecting axonal-transport within said living body of a said agent having a diagnostic marker moiety, preferably by generating an image of at least part of said body.

Although there are various rates and mechanisms of axonal transport, the fast anterograde and retrograde flows are carried out by motile proteins (kinesin and dynein respectively) which drag molecules and vesicles along the microtubules of the axoskeleton. The materials transported include not only structural and metabolic molecules, but also molecules sampled from the external environment of the axon terminus which are passed back up to the neuron cell body to inform it of the environment. Such signals include various trophic or growth factors originating in cells near the axon terminus which are endocytosed by the axon, encapsulated in lipid vesicles, and various trophic or growth factors originating in cells near the axon terminus which are endocytosed by the axon, encapsulated in lipid vesicles, and then passed up to the cell body for processing or analysis via the axonal transport system.

The rate of transport of a given substance is independent of electrical activity within a neuron but does vary with the type of molecule being transported. Anterograde axonal transport has a major fast and a slow component. The slow component is divided into “slow component a” and “slow component b” at rates of approximately 1 and 3 mm/day respectively. These slow components apparently reflect gradual structural repair and replacement of the subunits of the cytoskeleton and are not involved in the fast components important for tracer studies.

The fast component of transport demonstrates distinct maximal rates for anterograde (300-400 mm/day) and retrograde (150-300 mm/day) transport and some rates up to a meter/day have been reported. The maximal rates of transport apply to small membrane vesicles. Further, there are a variety of “waves” or distinct sets of slower transport rates exhibited in characteristic fashion by various molecules.

The existence of axonal transport (or ‘axoplasmic flow’) has been known for years and it has that certain foreign materials injected into muscle would be endocytosed (swallowed up) by the axon terminus and then subsequently be detectable in the neuron cell body.

da Vinci Robotic Prostatectomy

This platform allows physicians to perform a minimally invasive procedure that has been shown to offer patients significant benefits such as faster return to normal activity. The da Vinci Surgical System is the world's only robotic surgical platform designed to help physicians perform complex procedures through 1-2 cm incisions. It offers a physician superior visualization, enhanced dexterity and greater precision for the optimal performance of minimally invasive surgery.

The system consists of a surgeon's console that is linked to a patient-side surgical cart featuring four robotic arms that position and maneuver instruments and a 3D endoscopic camera. For most patients, da Vinci Prostatectomy offers substantially less pain and a much shorter recovery than traditional prostate surgery. Other advantages may include reduced need for blood transfusions; less scarring and less risk of infection. Moreover, recent studies suggest that da Vinci Prostatectomy may offer improved cancer control and a lower incidence of impotence and urinary incontinence.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N— and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N— and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamic, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention also provides nucleic acids encoding peptides, proteins, and antibodies of the invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), dimethylene-sulfoxide (—CH2-SO—CH2), dimethylene-sulfone (—CH2-SO2-CH2), 2′-O-alkyl, and 2′-deoxy2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

The present invention is directed to useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver an appropriate amount of the tracer molecule. Pharmaceutical compositions that are useful in the methods of the invention are generally administered locally, based on where the administration is and the tissue in which the visualization is to take place.

In addition to the appropriate compound, such pharmaceutical compositions may contain pharmaceutically-acceptable carriers and other ingredients known to enhance and facilitate drug administration.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

The invention also includes a kit comprising a compound of the invention and an instructional material which describes administering the composition to a subject. In another embodiment, this kit comprises a (preferably sterile) solvent suitable for dissolving or suspending the composition of the invention prior to administering the compound to the subject.

One of ordinary skill in the art will readily appreciate that the present invention is well adapted to carry out the objects and obtain the ends and advantages mentioned, as well as those inherent therein. The present invention may be embodied in other specific forms without departing from the spirit or essential attributes thereof.

Some examples of diseases and disorders which may be treated according to the methods of the invention are discussed herein. The invention should not be construed as being limited solely to these examples.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

The present invention provides composition, methods, and apparatuses for visualizing the cavernous nerves that directly control erectile function in real time in a living subject. The technique was developed to aid surgeons in nerve preservation during procedures, including, but not limited to, robotic radical prostatectomy, by allowing them to positively identify the anatomical position of the nerves in real time.

A feasibility study of intraoperative cavernous nerve imaging was performed. The aim of the study was to evaluate the novel nerve imaging protocol of the invention for its utility as a real-time surgical aid. The clinical application of primary interest was the prevention of damage to erectile nerves during prostatatic surgical interventions, resulting in a decreased risk of impotence following surgery.

Methods

The imaging equipment selected for use in the present invention was a Cell-vizio (purchased from Mauna Kea Technologies), an ultra-high resolution molecular imaging system that employs fibered confocal fluorescence microscopy technology. This system was specifically designed to visualize cellular structures and quantify molecular events in vivo and is composed of an autonomous Laser Scanning Unit, a range of fibered objectives made of tens of thousands of optical fibers, and a dedicated image processing and analysis software package. The system is capable of capturing images at a rate of 12 frames per second in living anesthetized animals. These images have a lateral resolution of the order of a micron, an axial resolution of 15 to 20 μm, a field of view up to 600 μm×500 μm, and can be obtained at depths up to 80 μm. The fibered probes have diameters ranging from 300 μm to 1.8 mm, allowing for access to the narrow spaces posterior to the prostate where the cavernous nerves are located.

To achieve organotypic nerve tracing from the penis, the retrograde nerve tracer AlexaFluor 488-coupled Cholera Toxin Subunit B was injected into the rat corpus cavernosum 24 hours prior to imaging. The cavernosal tissue was exposed with a midline incision and dissection through the peritoneal wall to the penile crura. Upon injection into the tissue, this marker is actively transported from the corpus cavernosum toward the central nervous system by nerves innervating the injected region. Employing retrograde nerve tracers for this organotypic nerve tracing technique allows one to positively identify cavernous nerves surrounding the prostate that are in fact innervating erectile tissue in the penis. The tracer used preferentially shows axons as opposed to other tracers that only identify cell bodies.

Fifty μl of AlexaFluor CTb at a concentration of 1 μg/μl was injected into each side of the penis. Larger volumes may be required in human studies as well as longer transit times.

Results

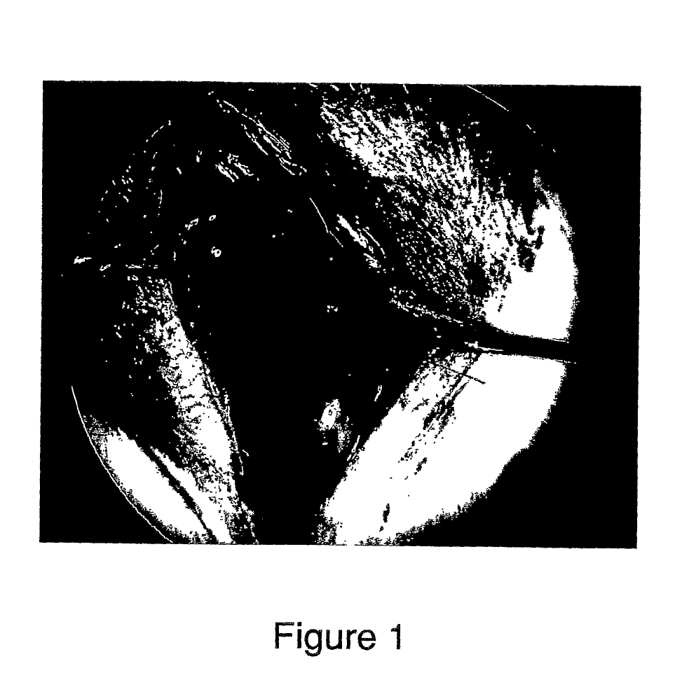

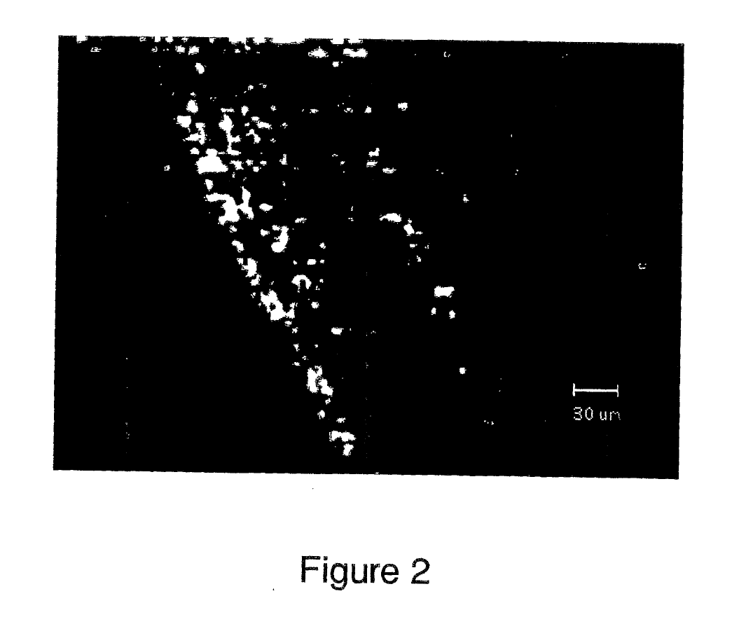

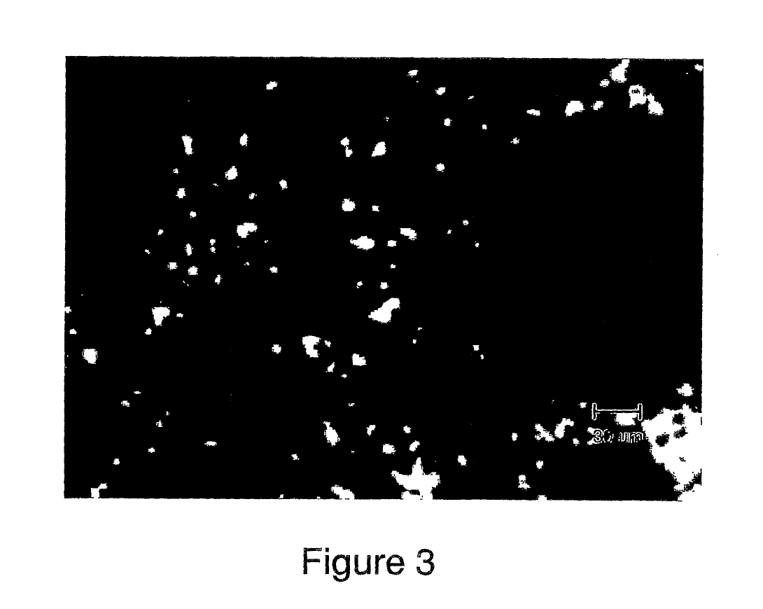

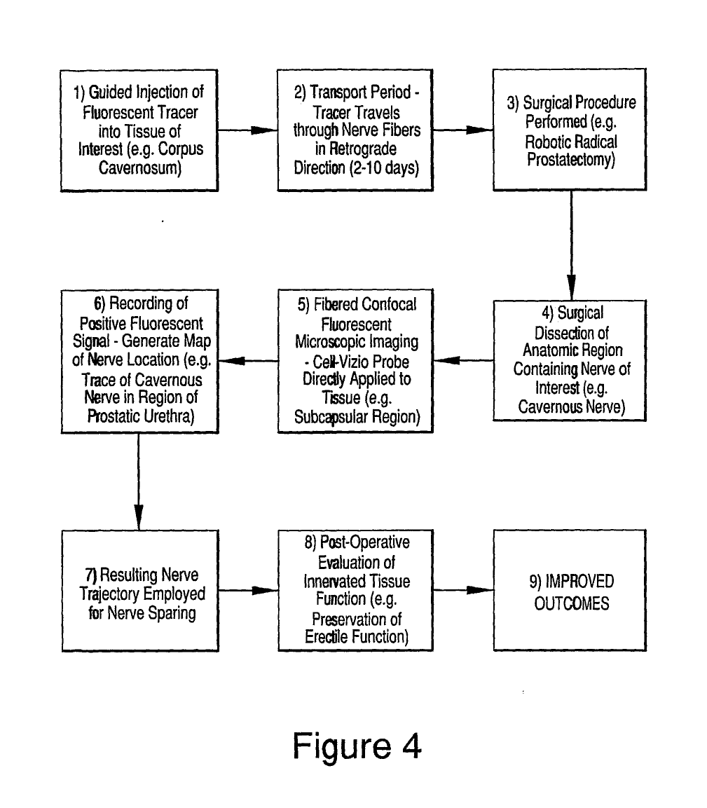

The progression of the fluorescent Cholera Toxin subunit through the axons of the cavernous nerves and their branches to the region of the prostate was detected by imaging the nerves using fibered confocal fluorescence microscopy. The miniaturization of the Cell-vizio systems allows one to visualize a positive fluorescent signal from cavernous nerves upstream of the injection site within a week of the tracer injection. The fluorescent images of the cavernous nerves, which were obtained with the Cell-vizio after performing retrograde nerve labeling in rats, provided useful structural information about the location of the nerves with respect to the prostate; such nerve localization is critical to sparing these nerves. The success of this imaging technique makes it feasible to identify and monitor delicate nerves during prostatic dissection in surgery, a priority for maintenance of sexual function in prostate cancer patients (see FIGS. 1-4).

Summary

The present invention relates to a novel invention for organotypic nerve tracing during surgery. Specifically, the technique disclosed herein has focused on identification of cavernous nerves during radical prostatectomy using retrograde tracers and fibered confocal fluorescence microscopy. This method allows precise identification of nerves directly responsible for erectile function of the penis. Positively identifying these nerves in the operative field enables surgeons to avoid severing or otherwise damaging them during surgery. As such, the present invention is of tremendous utility in performing nerve-sparing radical prostatectomy, with dramatically improved outcomes available to patients. However, the present invention has much greater utility than just surgical procedures involving nerves being identified during prostate surgery.

Intraoperative identification of functional nerves in virtually any anatomic site could be achieved using the invention. The present invention will also prove useful for cancer margin identification during surgery (again, this could be applied in many anatomic locations). This technique also has utility for nerve identification and tracing in experimental animal work.

The primary advantage of the present invention compared to every other method that has been used for nerve identification is that the tracing technique disclosed herein can be employed in a living subject with real-time results. These two characteristics are what allows the present invention to be used in the operating room, as opposed to conventional histological methods for nerve tracings, which are not only time consuming, but require removal of tissue for analysis, resulting in destruction of the nerve of interest. Such methods are obviously unsuited to use in patients. Because the methods of the present invention allow both identification and sparing of the nerves of interest, it can be widely applied in the clinical setting.

The disclosures of each and every patent, patent application, and publication cited herein are hereby incorporated by reference herein in their entirety. One of skill in the art will appreciate that the superiority of the compositions and methods of the invention relative to the compositions and methods of the prior art are unrelated to the physiological accuracy of the theory explaining the superior results.

Headings are included herein for reference and to aid in locating certain sections. These headings are not intended to limit the scope of the concepts described therein under, and these concepts may have applicability in other sections throughout the entire specification.

Other methods which were used but not described herein are well known and within the competence of one of ordinary skill in the art of clinical, chemical, cellular, histochemical, biochemical, molecular biology, microbiology and recombinant DNA techniques.

The description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

What is claimed is:

1. A method for identifying a nerve at a site of interest, said method comprising administering to a subject a pharmaceutical composition comprising at least one tracer at a site along the nerve distal to the location where the nerve is to be identified, wherein said tracer is taken up by the nerve and by retrograde transport moves to the site of interest of said nerve, and visualizing said nerve in microscopically at the site of interest, thereby identifying a nerve at a site of interest.

2. The method of claim 1, wherein said site of interest is revealed by a surgical procedure.

3. The method of claim 1, wherein said subject is a human.

4. The method of claim 1, wherein said site of interest is the prostate gland.

5. The method of claim 4, wherein said nerve comprises a cavernous nerve.

6. The method of claim 5, wherein said site of administration is selected from the group consisting of corpus cavernosum, corpus spongiosum, and crus of the penis.

7. The method of claim 6, wherein said tracer comprises AlexaFluor-488-conjugated cholera toxin subunit b.

8. The method of claim 7, wherein said tracer is visualized using fibered confocal fluorescent microscopy.

9. The method of claim 8, wherein said fibered confocal fluorescent microscopy is performed in conjunction with a robotic device to scan the site of interest.

10. The method of claim 9, wherein said nerve is visualized in real time.

11. The method of claim 9, wherein said method is performed to identify cavernous nerves during prostatectomy or cystectomy.

12. The method of claim 11, wherein identification of said nerve allows said nerve to be preserved during surgery.

13. The method of claim 1, wherein said method is used to identify the border of a tumor.

14. The method of claim 13, wherein said site of interest must be revealed by a surgical procedure.

15. The method of claim 13, wherein said subject is a human.

16. The method of claim 13, wherein said site of interest is the prostate gland.

17. The method of claim 16, wherein said tumor is a prostate tumor.

18. The method of claim 11, wherein said nerve comprises a cavernous nerve.

19. The method of claim 18, wherein said site of administration is selected from the group consisting of corpus cavernosum, corpus spongiosum, and crus of the penis.

20. The method of claim 19, wherein said tracer comprises AlexaFluor-488-conjugated cholera toxin subunit b, wherein said tracer is visualized using fibered confocal fluorescent microscopy, and wherein said fibered confocal fluorescent microscopy is performed in conjunction with a robotic device to scan the site of interest.
